(19) United States
(12) Patent Application Publication
(10) Pub. No.: US 2012/0213870 A1
(43) Pub. Date: Aug. 23, 2012

(54) COMPOSITION AND METHOD FOR TREATING LIVER DISEASE

(76) Inventor: Maher El-Aaser, Cairo (EG)

(21) Appl. No.: 13/397,812

(22) Filed: Feb. 16, 2012

Related U.S. Application Data
(60) Provisional application No. 61/443,772, filed on Feb. 17, 2011.

(51) Int. Cl. A61K 36/79; A61P 31/12; A61P 1/16

(52) U.S. Cl. 424/728

(57) ABSTRACT

A botanical composition for treating a patient with liver disease, e.g., Hepatitis C virus, is provided which includes dried fruit of Silybum sp., dried leaves of Cyanora sp., dried fruit of Ecballium sp., dried leaves of Centaurum sp., dried leaves of Taraxacum sp., gum of Commiphora sp., dried rhizomes of Rheum sp., dried rhizomes of Curcuma sp., dried leaves of Rosimarinus sp., dried fruit of Terminalia, sp., dried fruit of Schisandra sp., dried root of Panax sp., and dried root of Echinacea sp. The composition can be administered in any pharmaceutical dosage form suitable for enteral administration. A method for treating liver disease is provided which includes administering an effective amount of the botanical composition to a patient afflicted with liver disease. A method for manufacturing the botanical composition and unit dosage forms thereof is also provided.

### BACKGROUND

1. Technical Field

The present disclosure relates to treatment of liver disease such as Hepatitis C virus infections using botanical compositions.

2. Description of Related Art

Hepatitis C virus (HCV) is a significant cause of morbidity and mortality in humans. It has spread to more than 200 million people worldwide with steady increases in the number of cases each year. Despite recent progress, current therapies (first generation-nonspecific) remain inadequate for many patients since HCV is a virus that frequently mutates due to a lack of editing control during replication. Indeed, there are six different genotypes in at least 25% of the HCV nucleotide sequence, in addition to many subtypes.

Hepatitis C virus is an endemic flavin virus infection in Egypt (named Hepac virus) prevalent in about 20% of the population. Most cases of HCV in Egypt are classified as genotype 4. HCV is spread mainly through contact with blood (75% of cases) and less commonly (25% of cases) through community acquired routes which are still under investigation.

Herbal compositions have been used to treat or as an adjunct to treatment of Hepatitis C. See, e.g., U.S. Pat. No. 7,422,760 and Egypt Pat. No. 33673 (the '673 patent). The '673 patent is directed to a composition for treating Hepatitis C which includes Silybum marianum, Taraxacum officinale, Curcuma longa, Rosmarinus officinalis, Cynarascolymus, Centaurum erythrae, Commiphora molmol (also known as Commiphora myrrha), Ecballium elaterium, Rheum palmatum and Calenda officinalis.

Poor prognosis of chronic active hepatitis C virus patients is improved by more efficient screening examinations for earlier detection of non-diagnosed patients. Efficient management of HCV patients includes competent diet and drug restrictions, regular monthly colonic antiseptic and lavage measures and treatment of other liver co-infection.

There is a continuing need for effective treatments and adjuncts for treatment of liver diseases such as Hepatitis C virus infections.

### SUMMARY

A botanical composition for treating a patient is provided which includes dried fruit of Silybum sp., dried leaves of Cyanora sp., dried fruit of Ecballium sp., dried leaves of Centaurum sp., dried leaves of Taraxacum sp., gum of Commiphora sp., dried rhizomes of Rheum sp., dried rhizomes of Curcuma sp., dried leaves of Rosimarinus sp., dried fruit of Terminalia sp., dried fruit of Schisandra sp., dried root of Panax sp., and dried root of Echinacea sp. The composition can be administered in any suitable pharmaceutical dosage form for enteral administration, e.g., oral, rectal, etc administration.

A method for treating liver disease such as Hepatitis C virus infection is provided which includes administering to a patient with liver disease, an effective amount of a botanical composition which includes dried fruit of Silybum sp., dried leaves of Cyanora sp., dried fruit of Ecballium sp., dried leaves of Centaurum sp., dried leaves of Taraxacum sp., gum of Commiphora sp., dried rhizomes of Rheum sp., dried rhizomes of Curcuma sp., dried leaves of Rosimarinus sp., dried fruit of Terminali, sp., dried fruit of Schisandra sp., dried root of Panax sp., and dried root of Echinacea sp.

A method for manufacturing a botanical composition is provided which includes combining cleaned and ground: dried fruit of Silybum sp., dried leaves of Cyanora sp., dried fruit of Ecballium sp., dried leaves of Centaurum sp., dried leaves of Taraxacum sp., gum of Commiphora sp., dried rhizomes of Rheum sp., dried rhizomes of Curcuma sp., dried leaves of Rosimarinus sp., dried fruit of Terminalia sp., dried fruit of Schisandra sp., dried root of Panax sp., and dried root of Echinacea sp. The resulting powder may be divided into pharmaceutically acceptable unit dosage forms.

### BRIEF DESCRIPTION OF THE DRAWINGS

FIG. 1 is a bar graph depicting HCV load in patients as measured by polymerase chain reaction (PCR) over duration of treatment (“ttt”) with a composition according to the present disclosure.

FIG. 2 is a bar graph depicting HCV load in patients at the beginning of treatment with a composition according to the present disclosure and duration for complete remission or partial remission.

FIG. 3 is a bar graph depicting number of cases in relation to score of HCV activity before treatment and after treatment with a composition according to the present disclosure.

FIG. 4 is a bar graph depicting the relative amount of liver fibrosis in patients treated with a composition according to the present disclosure before treatment and after treatment.

### DETAILED DESCRIPTION OF PREFERRED EMBODIMENTS

The present botanical composition has been shown to be effective in the treatment of Hepatitis C viral infection in humans. Administration of the botanical composition to infected patients results in measurable decreases in serum levels of bilirubin, alananine transaminase (ALT) and aspartate transaminase (AST). Ultrasound imaging indicates reduction in size of enlarged livers after treatment. Polymerase chain reaction assays also show substantial reduction in viremia load.

A botanical composition for treating a patient includes dried fruit of Silybum sp., dried leaves of Cyanora sp., dried fruit of Ecballium sp., dried leaves of Centaurum sp., dried leaves of Taraxacum sp., dried flowers of Calendula sp., gum of Commiphora sp., dried rhizomes of Rheum sp., dried rhizomes of Curcuma sp., dried leaves of Rosimarinus sp., dried fruit of Terminalia, sp., dried fruit of Schisandra sp., dried root of Panax sp., and dried root of Echinacea sp.

Silybum sp. includes S. eburneum and S. marianum, each being suitable alone or in any combination herein, and is commonly known as milk thistle. The amount of Silybum sp., by weight of the composition, may range from about 10% to about 30%, e.g., about 20%. All percentages of components of the composition are given herein as a percentage by weight unless otherwise specified. Cyanora sp. includes C. cardunculas and scolymus, each being suitable alone or in any combination herein, is commonly known as artichoke, and ranges from about 5% to about 15%, e.g., about 10% of the composition. Ecballium sp. includes E. elaterium, is commonly known as squirting cucumber and ranges from about 2% to about 8%, e.g., about 5%, of the composition. Centaurum sp., includes C. pulchellum and C. erythrae, each being suitable alone or in any combination herein, is commonly known as common centaury, lesser centaury or European centaury and ranges from about 2% to about 8%, e.g., about 5%, of the composition. Taraxacum sp. includes T. officinale and T. erythrospermum, each being suitable alone or in combination herein, is commonly known as dandelion and ranges from about 3% to about 10%, e.g., about 7% of the composition. Calendula sp. includes C. arvensis, C. bicolor, C. eckerleinii, C. lanzae, C. maderensis, C. maritima, C. maroccana, C. meuselii, C. officinalis, C. stellata, C. suffruticosa and C. tripterocarpa, which are each suitable alone or in any combination herein, and is commonly known as marigold. The amount of Calendula sp. ranges from about 2% to about 8%, e.g., about 5%, of the composition. Commiphora sp. includes C. myrrha and C. gileadensis, each being suitable alone or in combination herein, is commonly known as myrrh and ranges from about 4% to about 12%, e.g., about 8%, of the composition. Rheum sp. includes R. rhabarbarum and R. palmatum, is commonly known as rhubarb and ranges from about 2% to about 8%, e.g., about 5%, of the composition. Curcuma sp. includes C. longa, is commonly known as turmeric and ranges from about 2% to about 8%, e.g., about 5%, of the composition. Rosmarinus sp. includes R. officinalis and R. eriocalyx, each being suitable alone or in combination herein, is commonly known as rosemary and ranges from about 2% to about 8%, e.g., about 5%, of the composition. Terminalia sp. includes T. chebula and ranges from about 5% to about 15%, e.g., about 10%, of the composition. Schisandra sp. include S. chinensis, S. glaucescens, S. rubriflora, and S. rubrifolia, each being suitable alone or in any combination herein. The amount of Schisandra sp. ranges from about 2% to about 8%, e.g., about 5%, of the composition. Panax sp. includes P. notoginseng, P. bipinnatifidus, P. ginseng, P. japonicus, P. quinquefoilius, P. vietnamensis, P. wangianus, P. zingiberensis, P. psuedoginseng, P. stipuleanatus and P. trifolius, each being suitable alone or in any combination herein. Panax sp. is commonly known as ginseng. The amount of Panax sp. ranges from about 2% to about 8%, e.g., about 5%, of the composition. Echinacea sp. includes E. angustifolia, E. atrorubens, E. laevigata, E. pallida, E. paradoxa, E. purpurea, E. sanguinea, E. simulata and E. tennesseensis, each being suitable alone or in any combination herein. Echinacea sp. is commonly known as echinacea or coneflower. The amount of Echinacea sp. ranges from about 2% to about 8%, e.g., about 5%, of the composition.

Techniques for cleaning, macerating, drying and milling botanicals such as those listed above are well known to those skilled in the art. For example, each of the above-identified botanicals is commercially available and may be separately washed, dried, pulverized, milled and combined in the proper ratios prior to compounding into a pharmaceutically acceptable dosage form. Techniques and devices for milling are described, e.g., in Remington's Pharmaceutical Sciences, Gennaro, A. R., 18th Edition, Mack Publishing Co., Easton, Pa., (1990) Chapt. 18. In embodiments, the botanicals may be milled in a stainless steel grinder having a sieve with 1 mm perforations. The ground components may then be passed through a 60 mesh sieve and any particles that do not pass through may be reground and sieved again to insure uniformity. The resulting powders may then be combined in the proper proportions and mixed, e.g., in a stainless steel rotating mixing tank to provide a uniform mixture.

Any pharmaceutically acceptable route of administration suitable for botanicals may be utilized. For example, any enteral route of administration, e.g., oral, nasal tube, GI tube, or rectal administration may be preferred. The mixture may be filled directly into capsules and/or combined with suitable pharmaceutical excipients and compounded into a pharmaceutically acceptable dosage form such as capsules, sachets, tablets or suppositories. Powders may be administered in tea bags for tea. Excipients may include disintegrants, lubricants, binders, diluents, preservatives, suspending agents, emulsifiers, flavoring agents and the like. Any excipients in the composition are excluded from the calculation of “weight percent” or “percent by weight.”

Suitable diluents illustratively include, either individually or in combination, lactose, including anhydrous lactose and lactose monohydrate; starches, including directly compressible starch and hydrolyzed starches; mannitol; sorbitol; xylitol; dextrose and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of amorphous cellulose and powdered cellulose; calcium carbonate; glycine; bentonite; polyvinylpyrrolidone; and the like. Such diluents, if present, may constitute in total about 5% to about 99%, about 10% to about 85%, or about 20% to about 80%, of the total weight of the composition.

Suitable disintegrants include, either individually or in combination, starches, including sodium starch glycolate and pregelatinized corn, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, xanthan, locust bean, karaya, pectin and tragacanth gums. Such disintegrants, if present, typically comprise in total about 0.2% to about 30%, about 0.2% to about 10%, or about 0.2% to about 5%, of the total weight of the composition.

Preservatives include sodium ascorbate, vitamin E (tocopherol), benzalkonium chloride, thimerisol and the like. One or more antioxidants, if present, are typically present in a composition of the invention in an amount of about 0.001% to about 5%, about 0.005% to about 2.5%, or about 0.01% to about 1%, by weight.

Binding agents or adhesives, particularly for tablet formulations preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches; celluloses such as, but not limited to, methylcellulose and carmellose sodium; alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose; and ethylcellulose. Such binding agents and/or adhesives, if present, may constitute in total about 0.5% to about 25%, about 0.75% to about 15%, or about 1% to about 10%, of the total weight of the composition.

Compositions of the invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene caprylic/capric mono- and diglycerides, polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Lubricants (including anti-adherents and/or glidants) include, either individually or in combination, glyceryl behapate; stearic acid and salts thereof, including magnesium (magnesium stearate), calcium and sodium stearates; hydrogenated vegetable oils; colloidal silica; talc; waxes; boric acid; sodium benzoate; sodium acetate; sodium fumarate; sodium chloride; DL-leucine; PEG; sodium oleate; sodium lauryl sulfate; and magnesium lauryl sulfate. Such lubricants, if present, may constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is an anti-adherent or glidant used, for example, to reduce formulation sticking to equipment surfaces and also to reduce static in the blend. Talc, if present, may constitute about 0.1% to about 10%, about 0.25% to about 5%, or about 0.5% to about 2%, of the total weight of the composition. Glidants can be used to promote powder flow of a solid formulation. Suitable glidants include colloidal silicon dioxide, starch, talc, tribasic calcium phosphate, powdered cellulose and magnesium trisilicate.

Flavoring agents include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents include, for example, acesulfame potassium (acesulfame K), alitame, aspartame, cyclamate, cylamate, dextrose, isomalt, maltitol, mannitol, neohesperidine DC, neotame, saccharin, sorbitol, stevia, sucralose, sucrose, tagatose, thaumatin, xylitol, high fructose corn syrup and the like.

Flavoring agents, sweetening agents, and/or colorants can be present in compositions of the invention in any suitable amount, for example about 0.01% to about 10%, about 0.1% to about 8%, or about 1% to about 5%, by weight.

The foregoing excipients can have multiple roles as is known in the art. For example, starch can serve as a filler as well as a disintegrant. The classification of excipients above is not to be construed as limiting in any manner. Excipients categorized in any manner may also operate under various different categories of excipients as will be readily appreciated by one of ordinary skill in the art.

In embodiments, a capsule contains 300 mg of powder as follows:

| | |
|---|---|
| Milk thistle fruit | 60 mg (20%) |
| Artichoke leaves | 30 mg (10%) |
| Squirting cucumber fruit | 15 mg (5%) |
| Common Centaury | 15 mg (5%) |
| Dandelion leaves | 21 mg (7%) |
| Calendula flower | 15 mg (5%) |
| Myrrh gum | 24 mg (8%) |
| Rhubarb rhizomes | 15 mg (5%) |
| Tumeric rhizomes | 15 mg (5%) |
| Rosemary leaves | 15 mg (5%) |
| Terminalia chebula fruit | 30 mg (10%) |
| Schisandra chinensis fruit | 15 mg (5%) |
| Panax ginseng root | 15 mg (5%) |
| Echinacea austifolia root | 15 mg (5%) |

The composition may also be suspended or emulsified for administration as a suspension or emulsion. Those skilled in the art are familiar with techniques for forming suspensions or emulsions. In this manner, if a patient cannot or will not swallow, e.g., a capsule or tablet, a liquid dosage form can be administered, e.g., by mouth, GI tube or intranasal feeding tube.

Effective dosages may range, e.g., from 300 mg to 5000 mg from one to four or more times daily. An “effective” amount is any amount of the botanical composition that inhibits or stops the progression of the viral infection, causes a detectable increase in liver function, decreases liver size, or decreases liver fibrosis. Greater or lesser amounts may be administered based on clinical experience and adjustments may be made by clinicians during the course of therapy. However, as will be apparent to those skilled in the art, the efficacy of a particular composition on a subject will be affected by a variety of factors including, but not limited to, the method of administration, the body mass and age of the subject, viral load, and the stage of the infection (e.g., acute or chronic). Therapy may be given for one week or more depending upon HCV severity and liver condition. For example, a course of treatment can range from 2 months to 24 months or longer.

The following Example is included for purposes of illustration and should not be considered as limiting the scope of the claims.

### Example

200 patients with chronic active Hepatitis C virus were treated according to the following protocol:
Diet and drug restrictions.
Regular monthly colonic antiseptic and lavage and full course of combined anti-bilharzial treatments in positive cases.
A botanical composition having the percentages of components given above for the 300 mg capsules is given to patients with their consent in a dose of 1800 mg every 12 hours before meals for a period ranging from 9 months to 24 months according to HCV severity and liver condition.
Monthly follow up for:

Laboratory analysis (Bilirubin, liver enzymes, renal function tests & complete blood picture).

Ultrasonography of the abdomen to examine the liver, portal vein, spleen and ascites.

A check of HCV-RNA PCR every 3 months to quantitatively assess improvement of blood viraemia.

A liver biopsy and histological examination every 6 months to assess improvement of liver insult. HCV activity scoring system ranges from a score of 1/9 up to a score of 9/9. A liver fibrosis grading system ranges from 01 to G4.

### Results:

The efficacy of the drug in the treatment of patients was evaluated as a comparison of the following values before and after treatment:

1—Laboratory Analysis

a) Serum bilirubin: the mean value of serum bilirubin level of the patients before treatment was 1.67 mg % (normally 0.2-1 mg %), and after treatment, the mean value of serum bilirubin was significantly lowered to the normal range 0.83 mg % (p value<0.001).

b) Serum liver enzymes (indicators of liver cell insult):

ALT (alanine transaminase): the mean value of serum ALT levels in patients before treatment was 91.13 units/ml (normally up to 40 units/ml), while after treatment the mean value of serum ALT levels was 26.56 units/ml which shows significant lowering to the normal range (P value<0.001).

AST (aspartate transaminase): the mean value of serum AST levels in patients before treatment was 108.68 units/ml (normally up to 37 units/ml), while after treatment the mean value of serum AST levels was 36.76 units/ml which shows significant lowering to the normal range (P value<0.001).

2—Ultrasonography (U/S)

a) Ultrasongraphic examination of liver condition (size & parenchymal affection) demonstrated that before the treatment, 73% of patients present with mild liver enlargement, 21.5% with moderate liver enlargement and 7% with shrunken cirrhotic liver. After the treatment there was significant improvement of liver condition: 62.5% of patients presented normal liver, 29% of patients showed mild enlargement, 1.5% of patients presented shrunken liver and 1% presented moderate enlargement.

N.B the rest of shrunken liver patients were probably missing cases.

b) Ultrasonographic examination of portal vein diameter (normally 8-12 mm).

The mean value of portal vein diameter before treatment was 13.38 mm, while after treatment there was significant improvement and normalization of portal vein diameter and pressure. The mean value of portal vein diameter decreased to 10.31 mm (normal diameter) (P value<0.001).

c) Ultrasonographic examination of spleen (size) before treatment showed that 58% of patients had moderate enlargement, 28% of patients had mild enlargement, 4% of patients had marked spleen enlargement, and 7% of patients had normal spleen size. After treatment, most patients showed significant improvement of spleen size: 51% of patients showed mild enlargement, 45% of patients had normal spleen size and only 1% had moderate spleen enlargement.

D) Ultrasonograpic examination for ascites before the treatment indicated 2% of patients with minimal ascites, 1% of cases showed moderate ascites and 1.5% of patients presented tense ascites. After the treatment, the patients showed significant improvement of ascites: only 1.5% of patients had minimal ascites.

3—HCV PCR (Polymerase Chain Reaction) Quantitative Assessment

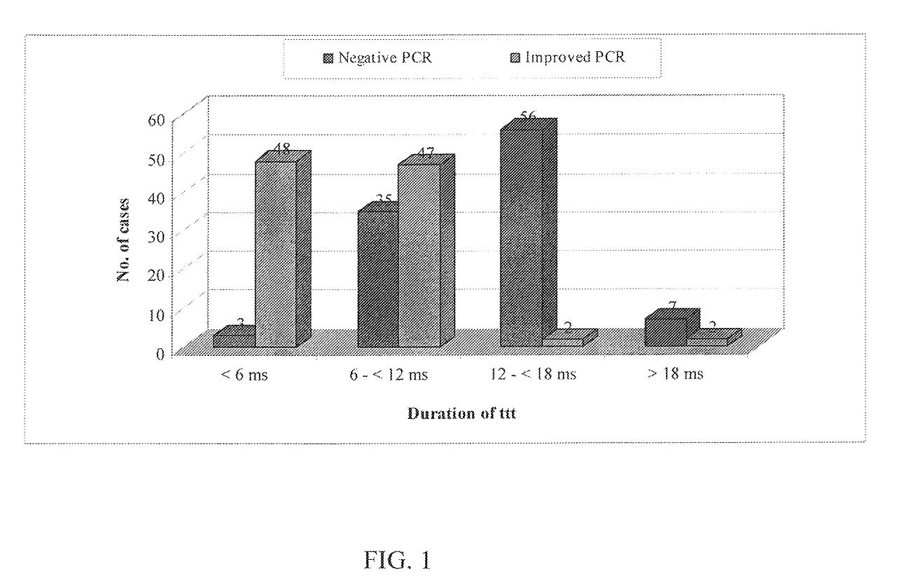

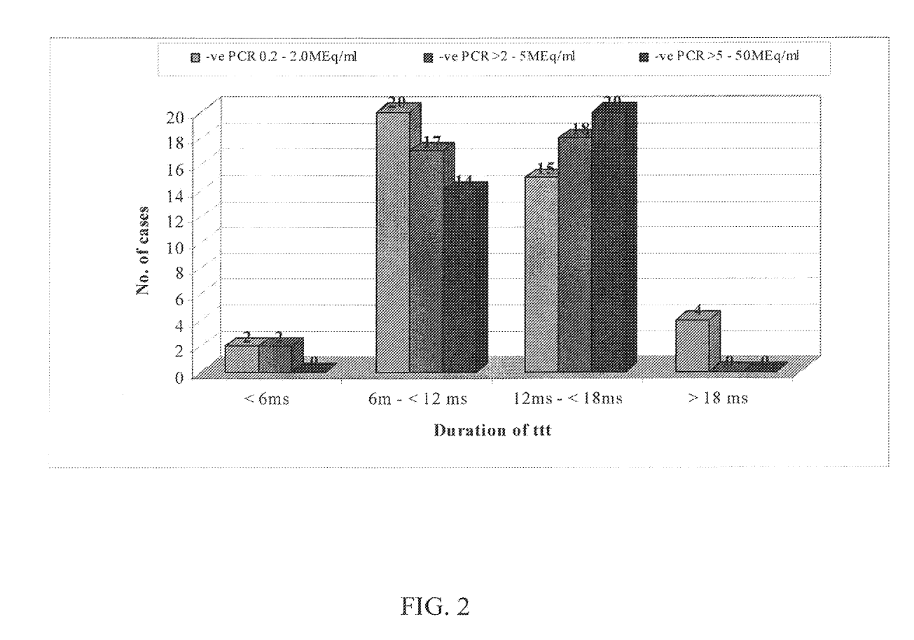

Three monthly HCV PCR Quantitative assessments of blood HCV viraemia of the patients showed complete remission (CR) of HCV viraemia in 101 patients (50.5%) by providing negative HCV PCR which was confirmed by repeating PCR, three times successively, two weeks apart between each negative result. The CR patients were classified into four groups according to the duration of treatment: 56 cases (28%) were cured in 1-1.5 years of treatment, 35 cases (17.5%) were cured in 1 year of treatment, 7 cases (3.5%) were cured in more than 1.5 years, and 3 cases (1.5%) were cured in less than 1 year. Without wishing to be bound by any theory, it appears as though the efficacy of the drug treatment and cure duration depends on the level of HCV viraemia at the beginning of treatment. There was partial remission (PR) of HCV viraemia in 99 cases (49.5%). 48 cases (24%) showed significant improvement in HCV PCR levels in less than 1 year, 47 cases (23.5%) were improved in 1 year, 2 cases (1%) were improved in 1-1½ years and 2 cases (1%) were improved in more than 1.5 years. As above, it appears as though the results depend on the level of HCV viraemia at the start of treatment. See FIG. 1). The relation between PCR-HCV viraemia at the beginning of treatment and duration of CR or PR is shown in FIG. 2.

4—Liver Biopsy and Histopathological Examination

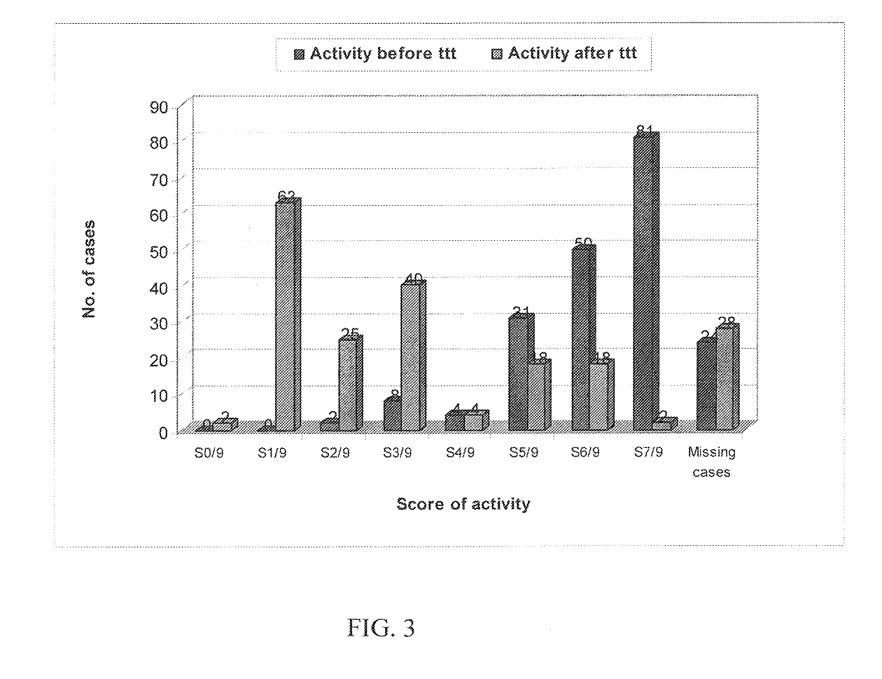

a) HCV activity scoring system (normal score is 0/9)—Prior to treatment, HCV activity showed that most of the patients, 131 cases (65.5%) had a high HCV activity score: (40.5% scored 7/9 and 25% scored 6/9), 35 cases (17.5%) had a moderate HCV activating score (15.5% scored 5/9 and 2% scored 4/9) and 10 cases (5%) had a mild HCV activity score (4% scored 3/9 and 1% scored 2/9). After treatment there was a significant improvement in HCV activity scores. 130 cases (65%) had a mild HCV activity score (31.5% scored 1/9; 20% scored 3/9, 12.5% scored 2/9 and 1% had complete arrested activity, i.e., scored 0/9). 22 cases (11%) had a moderate HCV activity score (9% scored 5/9 and 2% scored 4/9). 10% of patients had a high HCV activity score (9% scored 6/9 and 1% scored 7/9). See FIG. 3.

In summary, for all the patients, the mean value of HCV activity score before treatment was high, scoring 6.05/9, while after the treatment, the HCV activity score was significantly lowered to 2.68/9.

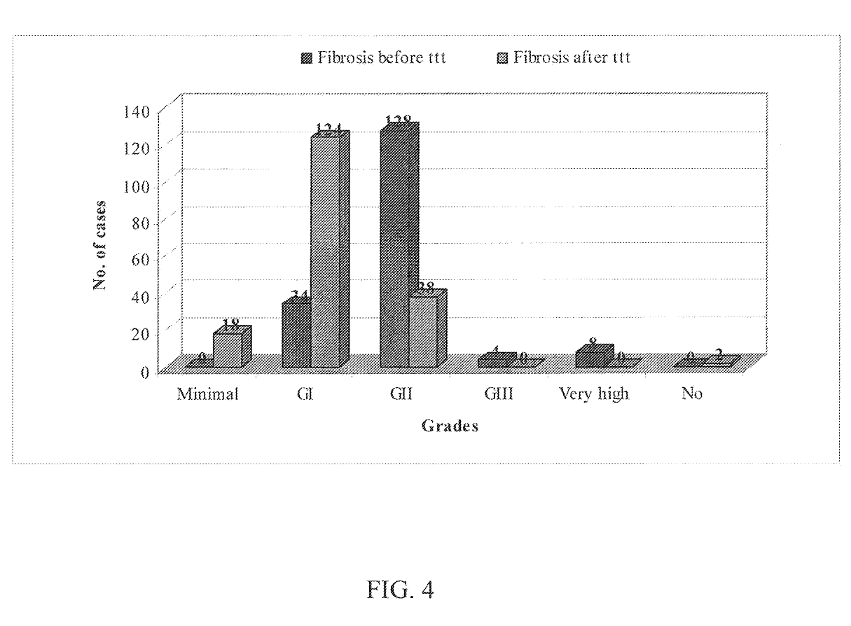

b) Liver fibrosis Grading system (normally no fibrosis)—Prior to treatment most patients, 128 cases (64%) had moderate liver fibrosis: G2, 34 cases (17%) had mild liver fibrosis: G1, 4 cases (2%) had high liver fibrosis: G3, and 8 cases (4%) had very high liver fibrosis: G4. After treatment there was a significant improvement in liver fibrosis. 124 cases (62%) had mild liver fibrosis G1, 38 cases (19%) had moderate liver fibrosis G2 and 20 cases (10%) had minimal or no liver fibrosis. There were no cases with high or very high liver fibrosis, thus indicating the success of the botanical composition in the treatment of HCV and its complications. See FIG. 4.

It should be understood that those skilled in the art can make modifications to the preferred embodiments described herein. For example, unit doses can have amounts other than 300 mg as described above. The botanical composition may be administered to patients who have liver conditions other than Hepatitis C. Those skilled in the art may envision additional modifications which are within the scope of the attached claims.

What is claimed is:

1. A botanical composition comprising dried fruit of Silybum sp.; dried leaves of Cyanora sp.; dried fruit of Ecballium sp.; dried leaves of Centaurum sp.; dried leaves of Taraxacum sp.; gum of Commiphora sp.; dried rhizomes of Rheum sp.; dried rhizomes of Curcuma sp.; dried leaves of Rosimarinus sp.; dried fruit of Terminalia, sp.; dried fruit of Schisandra sp.; dried root of Panax sp.; and dried root of Echinacea sp.

2. A botanical composition according to claim 1 wherein the amount of Silybum sp. ranges from about 10% to about 30% by weight; the amount of Cyanora sp. ranges from about 5% to about 15% by weight; the amount of Ecballium sp. ranges from about 2% to about 8% by weight; the amount of Centaurum sp. ranges from about 2% to about 8% by weight; the amount of Taraxacum sp. ranges from about 3% to about 10% by weight; the amount of Calendula sp. ranges from about 2% to about 8% by weight; the amount of Commiphora sp. ranges from about 4% to about 12% by weight; the amount of Rheum sp. ranges from about 2% to about 8% by weight; the amount of Curcuma sp. ranges from about 2% to about 8% by weight; the amount of Rosmarinus sp. ranges from about 2% to about 8% by weight; the amount of Terminalia sp. ranges from about 5% to about 15% by weight; the amount of Schisandra sp. ranges from about 2% to about 8% by weight; the amount of Panax sp, ranges from about 2% to about 8% by weight; the amount of Echinacea sp. ranges from about 2% to about 8% by weight.

3. A botanical composition according to claim 1 further comprising a pharmaceutically acceptable excipient.

4. A botanical composition according to claim 1 in a dosage form selected from the group consisting of capsule, tablet, suspension, emulsion and suppository.

5. A botanical composition according to claim 2 wherein the amount of Silybum sp. is about 20% by weight; the amount of Cyanora sp. is about 10% by weight; the amount of Ecballium sp. is about 5% by weight; the amount of Centaurum sp. is about 5% by weight; the amount of Taraxacum sp. is about 7% by weight; the amount of Calendula sp. is about 5% by weight; the amount of Commiphora sp. is about 8% by weight; the amount of Rheum sp. is about 5% by weight; the amount of Curcuma sp. is about 5% by weight; the amount of Rosmarinus sp. is about 5% by weight; the amount of Terminalia sp. is about 10% by weight; the amount of Schisandra sp. is about 5% by weight; the amount of Panax sp. is about 5% by weight; the amount of Echinacea sp. is about 5% by weight.

6. A botanical composition according to claim 5 wherein the Silybum sp. is S. marianum; the Cyanora sp. is C. scolymus; the Ecballium sp. is E. elaterium; the Centaurum sp. is C. erythrae; the Taraxacum sp. is T. officinale; the Calendula sp. is C. officinalis; the Commiphora sp. is C. myrrha; the Rheum sp. is R. palmatum; the Curcuma sp. is C. longa; the Rosmarinus sp. is R. officinalis; the Terminalia sp. is T. chebula; the Schisandra sp. is S. chinensis the Panax sp. is P. ginseng; and the Echinacea sp. is E. augustifolia, E. purpurea or both.

7. A method of treating liver disease in a patient comprising administering an effective amount of a composition according to claim 1 to a patient afflicted with liver disease.

8. A method of treating liver disease in a patient according to claim 7 wherein the composition is given twice a day.

9. A method of treating liver disease in a patient according to claim 8 wherein the total amount of the composition given in a day is 3600 mg.

10. A method of treating liver disease in a patient according to claim 7 wherein the liver disease is Hepatitis C virus.

11. A method of manufacturing a pharmaceutical dosage form of a botanical composition comprising:
cleaning, drying and milling dried fruit of Silybum sp., dried leaves of Cyanora sp., dried fruit of Ecballium sp., dried leaves of Centaurum sp., dried leaves of Taraxacum sp., gum of Commiphora sp., dried rhizomes of Rheum sp., dried rhizomes of Curcuma sp., dried leaves of Rosimarinus sp., dried fruit of Terminalia, sp., dried fruit of Schisandra sp., dried root of Panax sp., and dried root of Echinacea sp., to form powders;
combining the powders to form a uniform mixture; and
formulating the mixture into a pharmaceutically acceptable dosage form.

12. A method of manufacturing a pharmaceutical dosage form of a botanical composition according to claim 12 wherein the pharmaceutically acceptable dosage form is selected from the group consisting of capsule, tablet, suppository, sachet and tea bag.

13. A method of manufacturing a pharmaceutical dosage form of a botanical composition according to claim 11 wherein the pharmaceutically acceptable dosage form is a liquid.

14. A method of manufacturing a pharmaceutical dosage form of a botanical composition according to claim 13 wherein the liquid is a suspension or an emulsion.

15. A method of manufacturing a pharmaceutical dosage form of a botanical composition according to claim 11 further comprising adding a pharmaceutically acceptable excipient to the powders.

16. A method of manufacturing a pharmaceutical dosage form of a botanical composition according to claim 11 wherein the amount of Silybum sp. ranges from about 10% to about 30% by weight; the amount of Cyanora sp. ranges from about 5% to about 15% by weight; the amount of Ecballium sp. ranges from about 2% to about 8% by weight; the amount of Centaurum sp. ranges from about 2% to about 8% by weight; the amount of Taraxacum sp. ranges from about 3% to about 10% by weight; the amount of Calendula sp. ranges from about 2% to about 8% by weight; the amount of Commiphora sp. ranges from about 4% to about 12% by weight; the amount of Rheum sp. ranges from about 2% to about 8% by weight; the amount of Curcuma sp. ranges from about 2% to about 8% by weight; the amount of Rosmarinus sp. ranges from about 2% to about 8% by weight; the amount of Terminalia sp. ranges from about 5% to about 15% by weight; the amount of Schisandra sp. ranges from about 2% to about 8% by weight; the amount of Panax sp. ranges from about 2% to about 8% by weight; the amount of Echinacea sp. ranges from about 2% to about 8% by weight.

17. A method of manufacturing a pharmaceutical dosage form of a botanical composition according to claim 16 wherein the amount of Silybum sp. is about 20% by weight; the amount of Cyanora sp. is about 10% by weight; the amount of Ecballium sp. is about 5% by weight; the amount of Centaurum sp. is about 5% by weight; the amount of Taraxacum sp. is about 7% by weight; the amount of Calendula sp. is about 5% by weight; the amount of Commiphora sp. is about 8% by weight; the amount of Rheum sp. is about 5% by weight; the amount of Curcuma sp. is about 5% by weight; the amount of Rosmarinus sp. is about 5% by weight; the amount of Terminalia sp. is about 10% by weight; the amount of Schisandra sp. is about 5% by weight; the amount of Panax sp. is about 5% by weight; the amount of Echinacea sp. is about 5% by weight.

18. A method of manufacturing a pharmaceutical dosage form of a botanical composition according to claim 17 wherein the Silybum sp. is S. marianum; the Cyanora sp. is C. scolymus; the Ecballium sp. is E. elaterium; the Centaurum sp. is C. erythrae; the Taraxacum sp. is T. officinale; the Calendula sp. is C. officinalis; the Commiphora sp. is C. myrrha; the Rheum sp. is R. palmatum; the Curcuma sp. is C. longa; the Rosmarinus sp. is R. officinalis; the Terminalia sp. is T. chebula; the Schisandra sp. is S. S. chinensis the Panax sp. is P. ginseng; and the Echinacea sp. is E. augustifolia, E. purpurea or both.
